(19) United States
(12) Patent Application Publication
(10) Pub. No.: US 2024/0000556 A1
(43) Pub. Date: Jan. 4, 2024

(54) SYSTEM FOR DENTURE CLEANING AND DRUG INJECTION INTO A PATIENT'S MOUTH

(71) Applicants: Mohammad Hossein Rezadoost, Lahijan (IR); Ebrahim Ghorbandoost, Lahijan (IR); Naser Mohammadian Roushan, Lahijan (IR); Abbas Taghvaei, Lahijan (IR)

(72) Inventors: Mohammad Hossein Rezadoost, Lahijan (IR); Ebrahim Ghorbandoost, Lahijan (IR); Naser Mohammadian Roushan, Lahijan (IR); Abbas Taghvaei, Lahijan (IR)

(21) Appl. No.: 18/468,721

(22) Filed: Sep. 17, 2023

Related U.S. Application Data
(63) Continuation-in-part of application No. PCT/IB2022/052245, filed on Mar. 14, 2022.
(60) Provisional application No. 63/162,039, filed on Mar. 17, 2021.

(51) Int. Cl. A61C 17/16; A61C 19/06

(52) U.S. Cl. CPC A61C 17/16; A61C 19/063

(57) ABSTRACT

A denture cleaning system for cleaning and disinfecting a denture and a mouth of a user. The denture cleaning system includes a reservoir tank with an inlet and an outlet, a piston disposed slidably inside the reservoir tank, an actuating rod, an actuator, a nozzle, a check valve, one or more processors, and a sensor. The actuator is configured to urge the piston to move along the first axis and in a first direction inside the reservoir tank and, to thereby, discharge the mouthwash solution into the mouth of the user through the outlet of the reservoir tank. The one or more processors are configured to receive a first set of data from a user and send commands associated with movements of the actuating rod to the actuator.

### CROSS-REFERENCE TO RELATED APPLICATION

This application is a continuation-in-part of PCT/IB2022/052245 filed Mar. 14, 2022, and entitled “SYSTEM FOR DENTURE CLEANING AND DRUG INJECTION INTO A PATIENT'S MOUTH” which claims the benefit of priority from U.S. Provisional Patent Application Ser. No. 63/162,039, filed on Mar. 17, 2021, and entitled “INJECTOR DENTURE,” which both are incorporated herein by reference in their entirety.

### TECHNICAL FIELD

The present disclosure generally relates to artificial teeth and, particularly, relates to denture cleaning. The present disclosure, more particularly, relates to a denture cleaning device that is able to clean a denture without a need to removing the denture from the mouth of a patient. The disclosed system may further be used for drug injection into a patient's mouth.

### BACKGROUND ART

With the advancement of medical technology, a variety of medical apparatuses have been developed. For example, in dental medical technologies, artificial teeth and dentures have been developed and used. Dental implants typically connect dentures to the alveolar in a fixated configuration. Typically, the dentures are positioned in place and removed from the mouth of the patient by a procedure carried out by a dentist or another dentistry-professional. The fixated dentures are positioned in contact with the gums of the persons wearing dentures. In large size dentures (typically supporting more than one tooth), the contact with the gums is not continuous and homogenous throughout the surfaces of the dentures facing the gums.

In fixated dentures, on the surfaces of the gums and especially in areas in close vicinity to dental implants placed in the alveolar, the physiological conditions encourage the development of microorganisms. The development of microorganisms may cause bad-breath and/or infections. These infections may be severe so as to damage the connection between the implant and the alveolar as well as to cause pain and deteriorate the general health of the person having the implants in his or her mouth. To prevent the development of the microorganisms that thrive on the gums of patients wearing dentures, maintenance activities are required.

Typical maintenance activities, referred to as mechanical oral hygiene practices, are usually done by mechanical means such as, but not limited to, brushing, water squirting, and/or disinfecting solution which are limited in their effectiveness. The limited effectiveness may be due to the enclosure of the surfaces to be treated, especially areas in close vicinity to implants, between the gums and the dentures. Maintenance activities physically remove the microorganisms as well as disinfect the treated areas against remaining and new microorganisms. Maintenance activities may also include the removal of entrapped food particles that may serve as growth-media for microorganisms.

A classical method for the cleaning and disinfecting removable dentures, when worn as well as when not in use and for cleaning and disinfecting fixated dentures, is the spraying of a jet of water or a jet of a mouth-wash solution on the surfaces and/or side of the dentures by utilizing a device commonly known as a water flosser. On the other hand, denture users are often elderly who may be less inclined due to various limitations to rinse and clean their mouths. There is, therefore, a need for an effective and easy to use system for the cleaning and disinfecting mouth and gum surfaces of fixated dentures that help elderly, as well as dementia or Alzheimer patients, and people with similar limitations to wash out their mouth regularly at a routine basis. Such a system may also be used for injection of pharmaceutical suspension to older people and people associated with Alzheimer who use dentures, and are resistant to take their drugs due to depression.

### SUMMARY OF THE DISCLOSURE

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a denture cleaning system for cleaning and disinfecting a denture and a mouth of a user. In an exemplary embodiment, the denture cleaning system may include a reservoir tank, a piston, an actuating rod, an actuator, a nozzle, and a check valve.

In an exemplary embodiment, the reservoir tank may include an inlet and an outlet. In an exemplary embodiment, the reservoir tank may be configured to be disposed inside a denture of a user. In an exemplary embodiment, the reservoir tank may further be configured to receive and hold a mouthwash solution.

In an exemplary embodiment, the piston may be disposed slidably inside the reservoir tank. In an exemplary embodiment, the piston may be configured to move back and forth inside the reservoir tank and along a first axis. In an exemplary embodiment, the actuating rod may be connected to the piston. In an exemplary embodiment, a first end of the actuating rod may be attached to the piston.

In an exemplary embodiment, the actuator may be connected to the actuating rod. In an exemplary embodiment, a second end of the actuating rod may be attached to the actuator. In an exemplary embodiment, the actuator may be configured to urge the actuating rod to move back and forth along the first axis. In an exemplary embodiment, the nozzle may be connected to the outlet of the reservoir tank. In an exemplary embodiment, the nozzle may be in fluid communication with the reservoir tank through the outlet of the reservoir tank. In an exemplary embodiment, the nozzle may be configured to be attached to an inner side of the denture.

In an exemplary embodiment, the check valve may be connected to the inlet of the reservoir tank. In an exemplary embodiment, the check valve may be configured to be attached to an outer side of the denture. In an exemplary embodiment, the check valve may be configured to allow flowing of the mouthwash solution into the reservoir tank and through the inlet of the reservoir tank. In an exemplary embodiment, the check valve may further be configured to prevent the mouthwash solution discharge from the reservoir tank and through the inlet of the reservoir tank.

In an exemplary embodiment, the actuator may be configured to urge the piston to move along the first axis and in a first direction inside the reservoir tank and, to thereby, discharge the mouthwash solution into the mouth of the user through the outlet of the reservoir tank and the nozzle.

In an exemplary embodiment, the denture cleaning system may further include a controller board. In an exemplary embodiment, the controller board may include one or more processors. In an exemplary embodiment, the controller board may be configured to be disposed inside the denture of the user. In an exemplary embodiment, the controller board may be connected to the actuator through a connecting cable. In an exemplary embodiment, the one or more processors may be configured to send commands associated with movements of the actuating rod to the actuator.

In an exemplary embodiment, the one or more processors may further be configured to receive a first set of commands from a user through a wireless connection. In an exemplary embodiment, the one or more processors may further be configured to send a second set of commands to the actuator associated with movement of the actuating rod along the first axis and in the first direction based on the first set of commands. In an exemplary embodiment, the one or more processors may further be configured to send a third set of commands to the actuator associated with movement of the actuating rod along the first axis and in the second direction based on the first set of commands.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1 illustrates a denture cleaning system, consistent with one or more exemplary embodiments of the present disclosure. In an exemplary embodiment, denture cleaning system 100 may be implanted in a denture of a user.

FIG. 2 illustrates a denture cleaning system in a scenario in which the denture cleaning system is implanted in a denture of a user, consistent with one or more exemplary embodiments of the present disclosure.

FIG. 3 illustrates a denture cleaning system, consistent with one or more exemplary embodiments of the present disclosure.

FIG. 4 illustrates a denture cleaning system in a scenario in which a mouthwash solution is being injected into a reservoir tank by utilizing a syringe, consistent with one or more exemplary embodiments of the present disclosure.

FIG. 5 illustrates a denture cleaning system in a scenario in which the denture cleaning system is implanted in a denture of a user, consistent with one or more exemplary embodiments of the present disclosure.

FIG. 6 illustrates an example computer system in which an embodiment of the present disclosure, or portions thereof, may be implemented as computer-readable code, consistent with exemplary embodiments of the present disclosure.

### DESCRIPTION OF EMBODIMENTS

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

The present disclosure is directed to exemplary embodiments of a denture cleaning system for cleaning and disinfecting a denture and a mouth of a user. An exemplary denture cleaning system may include a reservoir tank, a piston disposed slidably inside the reservoir tank, an actuating rod, an actuator, a nozzle, and a filling valve. The reservoir tank may have an inlet and an outlet. An exemplary surgeon or a person trained appropriately may insert the reservoir tank inside the denture of the user. The filling valve may be connected to the inlet of the reservoir tank. The surgeon may charge the reservoir tank by injecting the mouthwash solution into the reservoir tank through the filling valve. The surgeon may use a syringe to inject the mouthwash solution into the reservoir tank.

The piston may be connected to the actuator by utilizing an actuating rod. Whenever it is needed to discharge the mouthwash solution into the mouth of the user, the actuator urges the actuating rod and the piston to move along a first axis and in a first direction so that the mouthwash is injected into the mouth through an outlet of the tank reservoir. The nozzle with a spray head is attached to the outlet of the reservoir tank. By utilizing the nozzle, when the mouthwash solution is injected into the mouth through the outlet of the reservoir tank, the mouthwash solution may be sprayed through the nozzle into the mouth of the user. The disclosed denture cleaning system also includes a processor which may be inserted into the denture as well. The processor may be connected to the actuator and control movements of the actuator. For example, the processor may urge the denture cleaning system to spray mouthwash solution into the mouth of the user at predetermined times.

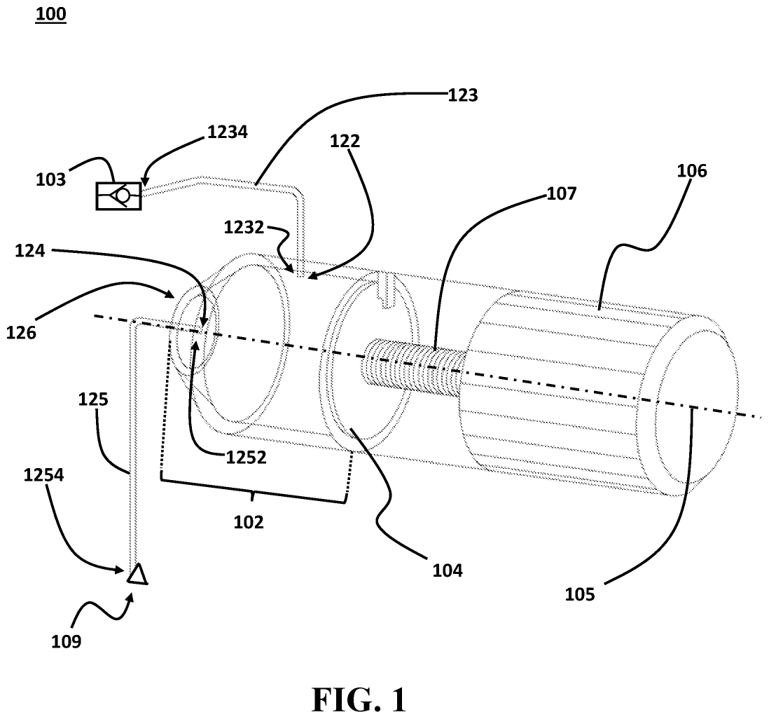

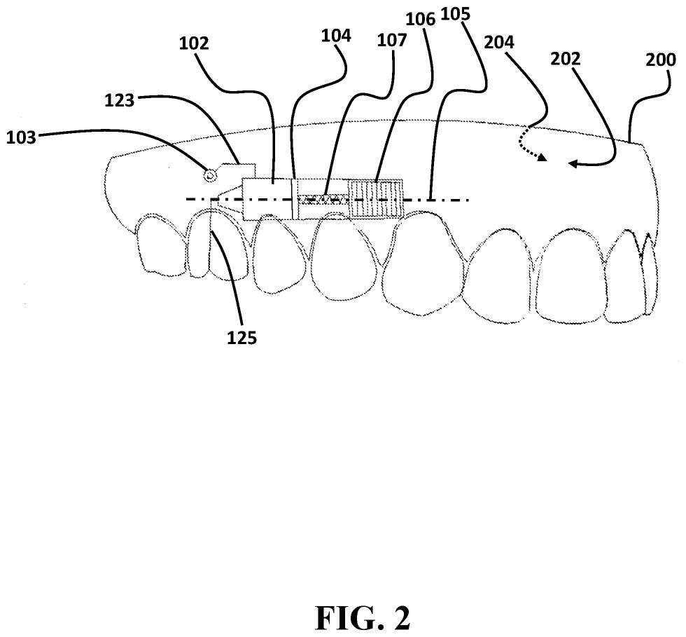

FIG. 1 shows a denture cleaning system 100, consistent with one or more exemplary embodiments of the present disclosure. In an exemplary embodiment, denture cleaning system 100 may be implanted in a denture of a user. In an exemplary embodiment, a surgeon may implant denture cleaning system 100 in a denture of a user. In an exemplary embodiment, a surgeon may implant denture cleaning system 100 in a denture of a user by inserting into a denture of a user. FIG. 2 shows denture cleaning system 100 in a scenario in which denture cleaning system 100 is implanted in a denture 200 of the user, consistent with one or more exemplary embodiments of the present disclosure. As shown in FIG. 1 and FIG. 2, in an exemplary embodiment, denture cleaning system 100 may include a reservoir tank 102. In an exemplary embodiment, a capacity of reservoir tank 102 may have a 1 milliliter capacity. In an exemplary embodiment, as illustrated reservoir tank may have a cylindrical body with a conical narrowing on one end with a flat surface. In an exemplary embodiment, reservoir tank 102 may include an inlet 122 and an outlet 124, that is inlet 122 may be an opening when a liquid may be inserted and outlet 124 may be an opening where a liquid might we forced out. In an exemplary embodiment, reservoir tank 102 may be disposed inside denture 200 of the user. In an exemplary embodiment, a surgeon may insert reservoir tank 102 into denture 200 of the user. In an exemplary embodiment, when a reservoir tank 102 is disposed inside denture 200 of the user, it may mean that reservoir tank 102 is implanted inside denture 200 of the user by a surgeon. In an exemplary embodiment, reservoir tank 102 may further receive and hold a mouthwash solution. In an exemplary embodiment, the mouthwash solution may be injected into reservoir tank 102 through inlet 122 of reservoir tank 102. In an exemplary embodiment, the mouthwash solution may be replaced with any other solution such as a disinfector or a drug.

As further shown in FIG. 1 and FIG. 2, in an exemplary embodiment, denture cleaning system 100 may further include a check valve 103. In an exemplary embodiment, check valve 103 may be a device which may be configured to control a flow of liquid that is inserted through inlet 122. In an exemplary embodiment, check valve 103 may be connected to inlet 122 of reservoir tank 102. In an exemplary embodiment, denture cleaning system 100 may further include an inlet tube 123. In an exemplary embodiment, inlet tube 123 may be interconnected between inlet 122 of reservoir tank 102 and check valve 103. In an exemplary embodiment, check valve 103 may be connected to inlet 122 of reservoir tank 102 by utilizing inlet tube 123. In an exemplary embodiment, inlet tube 123 may be a tube that may be made of for example a plastic or a metal material In an exemplary embodiment, a first end 1232 of inlet tube 123 may be attached to inlet 122 of reservoir tank 102. In an exemplary embodiment, a second end 1234 of inlet tube 123 may be attached to check valve 103. In an exemplary embodiment, inlet tube 123 may be disposed inside denture 200.

In an exemplary embodiment, check valve 103 may be utilized to manage flow of the mouthwash into reservoir tank 102 through inlet tube 123 and inlet 122 of reservoir tank 102. In other words, a surgeon may be able to inject mouthwash solution into reservoir tank 102 through check valve 103. In an exemplary embodiment, check valve 103 may further prevent the mouthwash solution from discharging from reservoir tank 102 and through inlet 122 of reservoir tank 102. In other words, the mouthwash solution may not be able to be discharged from inlet 122 of reservoir 102.

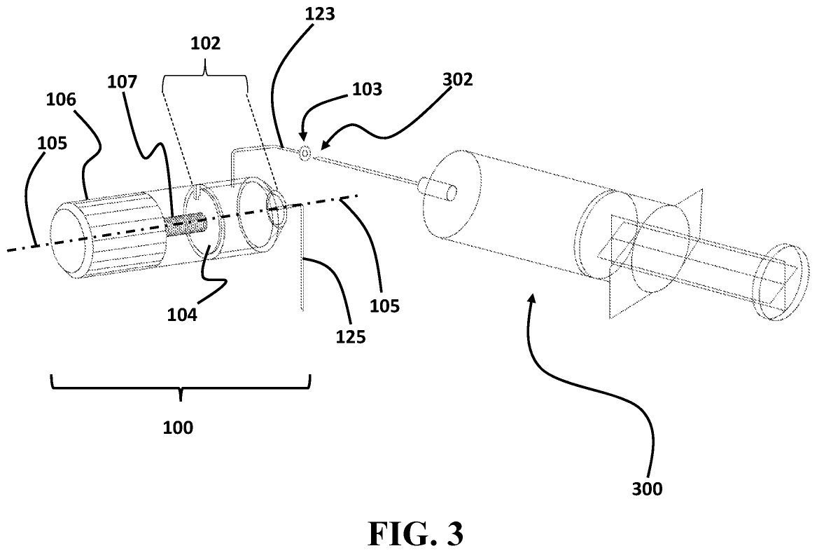

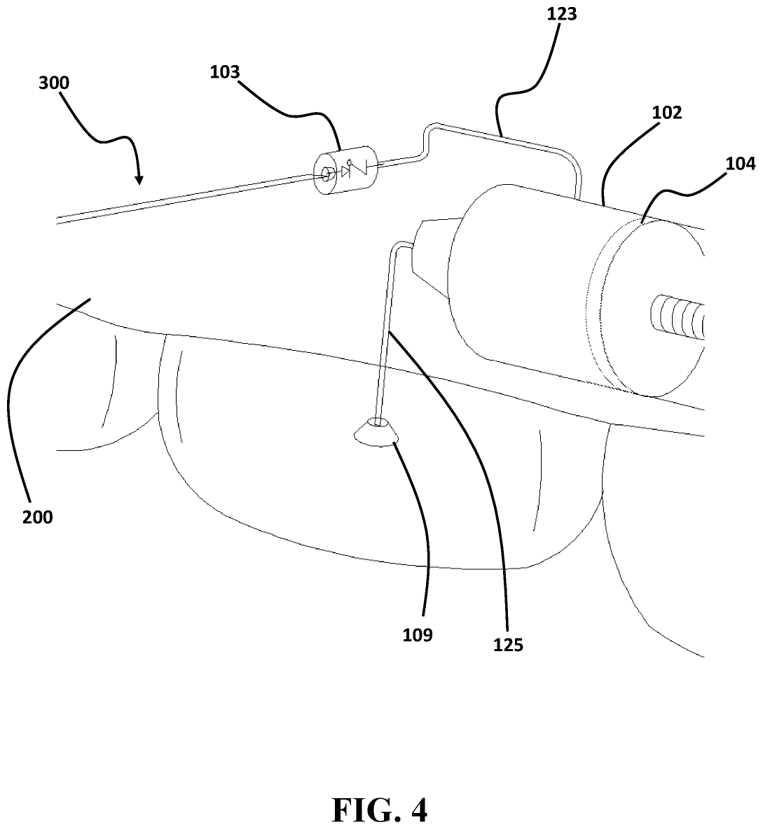

In an exemplary embodiment, a surgeon may inject the mouthwash solution into reservoir tank 102 through inlet 122 of reservoir tank 102. FIG. 3 shows denture cleaning system 100, consistent with one or more exemplary embodiments of the present disclosure. As shown in FIG. 3, in an exemplary embodiment, a surgeon may charge reservoir tank 102 by injecting the mouthwash solution into check valve 103 by utilizing a syringe 300. In an exemplary embodiment, check valve 103 may be attached to an outer side 202 of denture 200. In an exemplary embodiment, when check valve 103 is attached to outer side 202 of denture 200, the surgeon may easily insert a head 302 of syringe 300 into check valve 103 and inject the mouthwash solution into reservoir tank 102. FIG. 4 shows denture cleaning system 100 in a scenario in which the mouthwash solution is being injected into reservoir tank 102 by utilizing syringe 300, consistent with one or more exemplary embodiments of the present disclosure.

As further shown in FIG. 1, FIG. 2, and FIG. 3, in an exemplary embodiment, system 100 may further include a piston 104. In an exemplary embodiment, piston 104 may have a circular flat surface corresponding to cylindrical shape of reservoir tank 102. In an exemplary embodiment, piston 104 may be disposed slidably inside reservoir tank 102. In an exemplary embodiment, when piston 104 is disposed slidably inside reservoir tank 102, it may mean that piston 104 is disposed inside reservoir tank 102 in such a way that piston 104 is able to move back and forth inside reservoir tank 102, that is, across a main axis (along a longest distance in middle) of reservoir tank 102. In an exemplary embodiment, piston 104 may move back and forth inside reservoir tank 102 and along a first axis 105. In an exemplary embodiment, first axis 105 may coincide with a main longitudinal axis of reservoir tank 102. In an exemplary embodiment, system 100 may further include an actuator 106 and an actuating rod 107. In an exemplary embodiment, actuating rod 107 may be interconnected between piston 104 and actuator 106. In an exemplary embodiment, a first end 172 of actuating rod 107 may be attached to piston 104. In an exemplary embodiment, a second end 174 of actuating rod 107 may be attached to actuator 106. In an exemplary embodiment, actuator 106 may be configured to actuate actuating rod 107 to move back and forth along first axis 105. In an exemplary embodiment, when actuating rod 107 moves back and forth along first axis 105, piston 104 may also move accordingly along first axis 105. In an exemplary embodiment, when actuator 106 urges actuating rod 107 to move along first axis 105 and in a first direction 152, actuating rod 107 and piston 104 may move along first axis 105 and in first direction 152. In an exemplary embodiment, when actuator 106 urges actuating rod 107 to move along first axis 105 and in a second direction 154, actuating rod 107 and piston 104 may move along first axis 105 and in second direction 154.

In an exemplary embodiment, system 100 may further include a nozzle 109. In an exemplary embodiment, nozzle 109 may be connected to outlet 124 of reservoir tank 102. In an exemplary embodiment, outlet 124 of reservoir tank 102 may be at a first end 126 of reservoir tank 102. In an exemplary embodiment, nozzle 109 may be in fluid communication with reservoir tank 102. In an exemplary embodiment, nozzle 109 may be an atomizing nozzle. In an exemplary embodiment, an atomizing nozzle may refer to a nozzle that may atomize fluids by creating a very fine spray. In an exemplary embodiment, nozzle 109 may be be attached to an inner side 204 of denture 200. In an exemplary embodiment, denture cleaning system 100 may further include an outlet tube 125. In an exemplary embodiment, outlet tube 125 may be interconnected between outlet 124 of reservoir tank 102 and nozzle 109. In an exemplary embodiment, a first end 1252 of outlet tube 125 may be attached to outlet 124 of reservoir tank 102. In an exemplary embodiment, a second end 1254 of outlet tube 125 may be attached to nozzle 109.

In an exemplary embodiment, when piston 104 moves inside reservoir tank 102 along first axis 105 and in second direction 154, the mouthwash solution inside reservoir tank 102 may be discharged to the mouth of the user through outlet 124 of reservoir tank 102, outlet tube 125, and nozzle 109. In an exemplary embodiment, actuator 106 may move actuating rod 107 and piston 104 along first axis 105 and in first direction 152 and, to thereby, may discharge the mouthwash solution through outlet 124 of reservoir tank 102.

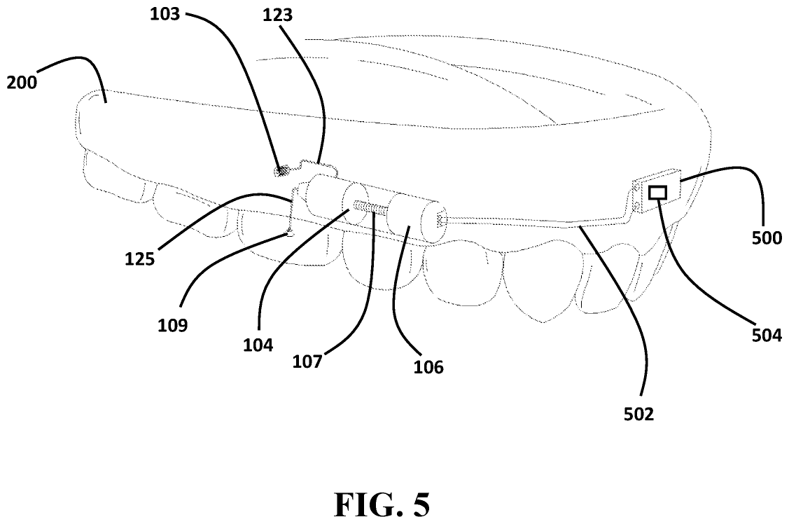

FIG. 5 shows denture cleaning system 100 in a scenario in which denture cleaning system 100 is implanted in denture 200 of the user, consistent with one or more exemplary embodiments of the present disclosure. As shown in FIG. 5, in an exemplary embodiment, denture cleaning system 100 may further include a controller board 500. In an exemplary embodiment, controller board 500 may be connected to actuator 106 through a connecting cable 502. In an exemplary embodiment, controller board 500 may include one or more processors 504. In an exemplary embodiment, controller board 500 may be disposed inside denture 200. In an exemplary embodiment, a surgeon may implant controller board 500 inside denture 200. In an exemplary embodiment, one or more processors 504 may send commands associated with movements of actuating rod 107 to actuator 106.

In an exemplary embodiment, one or more processors 504 may further receive a first set of commands from a user through a wireless connection. In an exemplary embodiment, one or more processors 504 may further send a second set of commands to actuator 106 associated with movement of actuating rod 107 along first axis 105 and in first direction 152 based on the first set of commands. In an exemplary embodiment, one or more processors 504 may further send a third set of commands to actuator 106 associated with movement of actuating rod 107 along first axis 105 and in second direction 154 based on the first set of commands.

In an exemplary embodiment, denture cleaning system 100 may further include a sensor disposed inside reservoir tank 102. In an exemplary embodiment, the said sensor may send a first set of data to one or more processors 504.

In an exemplary embodiment, one or more processors 504 may be in a wireless communication with a user interface module. For example, one or more processors 504 may be connected to an android application on mobile of a user through a wireless connection such as Bluetooth. In an exemplary embodiment, the user may send the first set of commands to one or more processors 504 by utilizing his or her mobile. Also, one or more processors 504 may send a second set of data to the mobile of the user. In an exemplary embodiment, the second set of data may be based on the first set of data which one or more processors 504 may receive from the sensor. For example, one or more processors 504 may send a data to the mobile of the user that identifies the amount of the mouthwash solution in reservoir tank 102. Once the mobile of the user receives this data, the android application on the mobile may show the amount of the mouthwash solution to the user. Then, the user may be able to refill reservoir tank 102 whenever it is needed. For example, when the amount of the mouthwash solution in reservoir tank 102 becomes less than a threshold, the user may be advised of it through his/her phone and then may refill reservoir tank 102.

The android application on the mobile of the user may control operations and functions of one or more processors 504. In this application, the charge level of batteries of the controller board may also be displayed. In an exemplary embodiment, the application may be designed in such a way that it is used to increase or decrease the duration of pump operation and subsequently mouthwash solution injection. In an exemplary embodiment, different times for injection may be may also be selected using the application. At these times, the application may automatically command an injection. In an exemplary embodiment, the application may also have a spraying option by which the mouthwash solution in reservoir tank 102 may immediately be discharged into the mouth of the user at a set amount.

In an exemplary embodiment, denture cleaning system 100 may also be used for drug injection into a patient's mouth. For example, for patients who are suffered from Alzheimer or for any other reason may forget to eat their drugs, denture cleaning system 100 may be used to inject their drug at a specific time to their mouths.

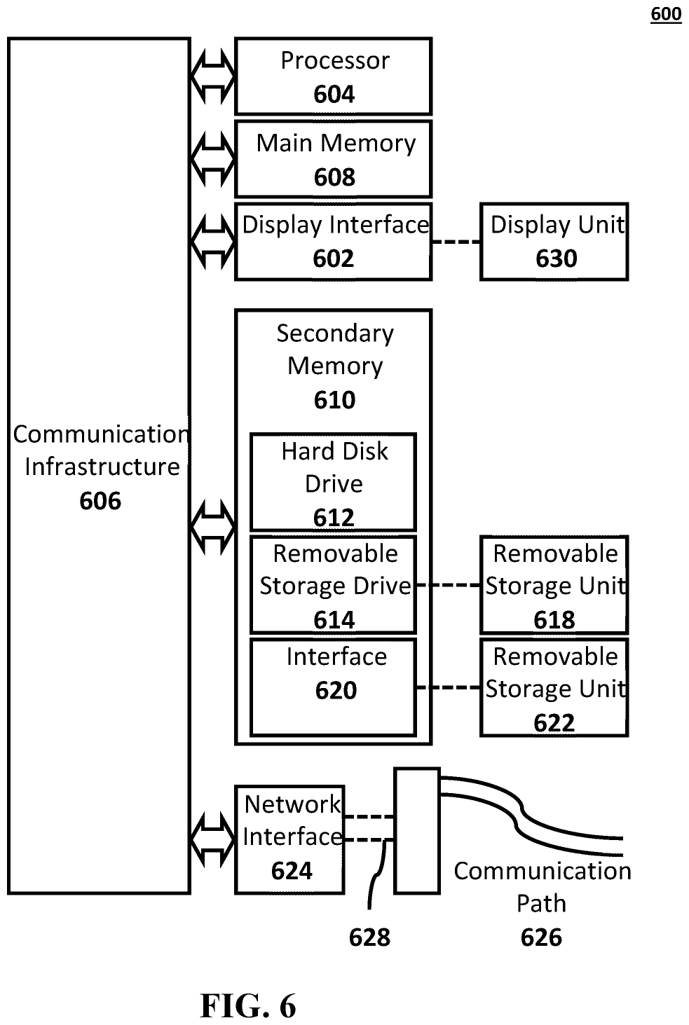

FIG. 6 shows an example computer system 600 in which an embodiment of the present disclosure, or portions thereof, may be implemented as computer-readable code, consistent with exemplary embodiments of the present disclosure. For example, one or more processors 504 may be implemented in computer system 600 using hardware, software, firmware, tangible computer readable media having instructions stored thereon, or a combination thereof and may be implemented in one or more computer systems or other processing systems.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the disclosure is described in terms of this example computer system 600. After reading this description, it will become apparent to a person skilled in the relevant art how to implement the disclosure using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 604 may be a special purpose or a general-purpose processor device. As will be appreciated by persons skilled in the relevant art, processor device 604 may also be a single processor in a multi-core/multiprocessor system, such system operating alone, or in a cluster of computing devices operating in a cluster or server farm. Processor device 604 may be connected to a communication infrastructure 606, for example, a bus, message queue, network, or multi-core message-passing scheme.

In an exemplary embodiment, computer system 600 may include a display interface 602, for example a video connector, to transfer data to a display unit 630, for example, a monitor. Computer system 600 may also include a main memory 608, for example, random access memory (RAM), and may also include a secondary memory 610. Secondary memory 610 may include, for example, a hard disk drive 612, and a removable storage drive 614. Removable storage drive 614 may include a floppy disk drive, a magnetic tape drive, an optical disk drive, a flash memory, or the like. Removable storage drive 614 may read from and/or write to a removable storage unit 618 in a well-known manner. Removable storage unit 618 may include a floppy disk, a magnetic tape, an optical disk, etc., which may be read by and written to by removable storage drive 614. As will be appreciated by persons skilled in the relevant art, removable storage unit 618 may include a computer usable storage medium having stored therein computer software and/or data.

In alternative implementations, secondary memory 610 may include other similar means for allowing computer programs or other instructions to be loaded into computer system 600. Such means may include, for example, a removable storage unit 622 and an interface 620. Examples of such means may include a program cartridge and cartridge interface (such as that found in video game devices), a removable memory chip (such as an EPROM, or PROM) and associated socket, and other removable storage units 622 and interfaces 620 which allow software and data to be transferred from removable storage unit 622 to computer system 600.

Computer system 600 may also include a communications interface 624. Communications interface 624 allows software and data to be transferred between computer system 600 and external devices. Communications interface 624 may include a modem, a network interface (such as an Ethernet card), a communications port, a PCMCIA slot and card, or the like. Software and data transferred via communications interface 624 may be in the form of signals, which may be electronic, electromagnetic, optical, or other signals capable of being received by communications interface 624. These signals may be provided to communications interface 624 via a communications path 626. Communications path 626 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 618, removable storage unit 622, and a hard disk installed in hard disk drive 612. Computer program medium and computer usable medium may also refer to memories, such as main memory 608 and secondary memory 610, which may be memory semiconductors (e.g. DRAMs, etc.).

Computer programs (also called computer control logic) are stored in main memory 608 and/or secondary memory 610. Computer programs may also be received via communications interface 624. Such computer programs, when executed, enable computer system 600 to implement different embodiments of the present disclosure as discussed herein. In particular, the computer programs, when executed, enable processor device 604 to implement the processes of the present disclosure. Accordingly, such computer programs represent controllers of computer system 600. Where an exemplary embodiment of the present disclosure may be implemented using software, the software may be stored in a computer program product and loaded into computer system 600 using removable storage drive 614, interface 620, and hard disk drive 612, or communications interface 624.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Ends 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective spaces of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A denture cleaning system for cleaning and disinfecting a denture and a mouth of a user, the denture cleaning system comprising:
a cylindrical shaped reservoir tank with a capacity of 1 milliliter, the reservoir tank comprising an inlet and an outlet, the reservoir tank configured to:
be disposed inside a denture of a user; and
receive and hold a mouthwash solution;
a piston disposed slidably inside the reservoir tank, the piston configured to move back and forth inside the reservoir tank and along a first axis;
an actuating rod connected to the piston, a first end of the actuating rod attached to the piston;
an actuator connected to the actuating rod, a second end of the actuating rod attached to the actuator, the actuator configured to urge the actuating rod to move back and forth along the first axis;
a nozzle connected to the outlet of the reservoir tank, the nozzle in fluid communication with the reservoir tank through the outlet of the reservoir tank, the nozzle configured to be attached to an inner side of the denture;
a check valve connected to the inlet of the reservoir tank, the check valve configured to be attached to an outer side of the denture, the check valve configured to:
allow flow of the mouthwash solution into the reservoir tank and through the inlet of the reservoir tank; and
prevent the mouthwash solution discharge from the reservoir tank and through the inlet of the reservoir tank;
a controller board, the controller board comprising one or more processors; the controller board configured to be disposed inside the denture of the user; the controller board connected to the actuator through a connecting cable; the one or more processors are configured to send commands associated with movements of the actuating rod to the actuator; and
a sensor disposed inside the reservoir tank, the sensor configured to send a first set of data to the one or more processors, the first set of data associated with an amount of the mouthwash solution in the reservoir tank;
wherein:
the actuator is configured to discharge the mouthwash solution into the mouth of the user through the outlet of the reservoir tank and the nozzle by urging the piston to move along the first axis and in a first direction inside the reservoir tank and;
the one or more processors are further configured to:
receive a first set of commands from a user through a wireless connection;
send a second set of commands to the actuator associated with movement of the actuating rod along the first axis and in the first direction based on the first set of commands; and
send a third set of commands to the actuator associated with movement of the actuating rod along the first axis and in the second direction based on the first set of commands.

2. A denture cleaning system for cleaning and disinfecting a denture and a mouth of a user, the denture cleaning system comprising:
a reservoir tank, the reservoir tank comprising an inlet and an outlet, the reservoir tank configured to:
be disposed inside an opening within a denture of a user; and
receive and hold a mouthwash solution;
a piston disposed slidably inside the reservoir tank, the piston configured to move back and forth inside the reservoir tank and along a first axis;
an actuating rod connected to the piston, a first end of the actuating rod attached to the piston;
an actuator connected to the actuating rod, a second end of the actuating rod attached to the actuator, the actuator configured to urge the actuating rod to move back and forth along the first axis;
a nozzle connected to the outlet of the reservoir tank, the nozzle in fluid communication with the reservoir tank through the outlet of the reservoir tank, the nozzle configured to be attached to an inner side of the denture; and
a check valve connected to the inlet of the reservoir tank, the check valve configured to be attached to an outer side of the denture, the check valve configured to:
allow flowing of the mouthwash solution into the reservoir tank and through the inlet of the reservoir tank; and
prevent the mouthwash solution discharge from the reservoir tank and through the inlet of the reservoir tank;
wherein the actuator is configured to discharge the mouthwash solution into the mouth of the user through the outlet of the reservoir tank and the nozzle by urging the piston to move along the first axis and in a first direction inside the reservoir tank and.

3. The denture cleaning system of claim 2, further comprising a controller board, wherein:
the controller board comprises one or more processors;
the controller board is configured to be disposed inside the denture of the user;
the controller board is connected to the actuator through a connecting cable; and
the one or more processors are configured to send commands associated with movements of the actuating rod to the actuator.

4. The denture cleaning system of claim 3, wherein the one or more processors are further configured to:
receive a first set of commands from a user through a wireless connection;
send a second set of commands to the actuator associated with movement of the actuating rod along the first axis and in the first direction based on the first set of commands; and
send a third set of commands to the actuator associated with movement of the actuating rod along the first axis and in the second direction based on the first set of commands.

5. The denture cleaning system of claim 4, wherein the nozzle is an atomizing nozzle.

6. The denture cleaning system of claim 5, further comprising a sensor disposed inside the reservoir tank, the sensor configured to send a first set of data to the one or more processors, the first set of data associated with an amount of the mouthwash solution in the reservoir tank.

7. The denture cleaning system of claim 6, wherein a capacity of the reservoir tank is 1 milliliter.
